(19) United States
(12) Patent Application Publication
(10) Pub. No.: US 2008/0254091 A1
(43) Pub. Date: Oct. 16, 2008

(54) Multi-Layered Antiadhesion Barrier

(76) Inventors: Young-Woo Lee, Gyeonggi-do (KR); Bo-Young Chu, Gyeonggi-do (KR)

Correspondence Address: IPLA P.A., 3580 WILSHIRE BLVD., 17TH FLOOR, LOS ANGELES, CA, 90010, US

(21) Appl. No.: 12/065,713

(22) PCT Filed: Jul. 14, 2006
(86) PCT No.: PCT/KR06/02782

§ 371 Date: Mar. 4, 2008

(30) Foreign Application Priority Data
Sep. 5, 2005 (KR) 10-2005-0082189
Jul. 14, 2006 (KR) PCT/KR2006/002782

(51) Int. Cl. A61L 31/04; B32B 5/02; B32B 5/18; B05D 3/00; B05D 1/00; A61K 38/48; A61K 38/57; A61K 31/727; A61K 38/49

(52) U.S. Cl. 424/423; 428/221; 428/318.6; 427/322; 427/457; 424/94.64; 514/12; 514/56

(57) ABSTRACT

The present invention relates to a multi-layered anti-adhesion barrier, particularly to a multi-layered anti-adhesion barrier comprising a nanofibrous structured base layer electrospun from a hydrophobic, biodegradable, biocompatible polymer and a polymer layer formed by coating a hydrophilic, biooriginated polymer on the base layer, and a method for the preparing the same. The multi-layered anti-adhesion barrier of the present invention can solve the problems of the conventional gel, solution, sponge, film or nonwoven type anti-adhesion systems, including adhesion to tissues or organs, flexibility, physical strength, ease of handling (ease of folding and bending), etc., offers improved user convenience. With a nanofibrous structure, the multi-layered anti-adhesion barrier of the present invention effectively blocks the infiltration or migration of blood and cells and promotes the healing of wounds. It is not torn or broken when folded or rolled and can be easily handled using small surgical instruments. Thus, it can minimize a foreign body reaction when used in various surgical operations.

### TECHNICAL FIELD

The present invention relates to a multi-layered anti-adhesion barrier, and more particularly to a multi-layered anti-adhesion barrier having improved anti-adhesion properties by solving the problems of the conventional gel, solution, sponge, film or nonwoven type anti-adhesion systems, including adhesion to tissues or organs, flexibility, physical strength, ease of handling (ease of folding and bending), etc., offers improved user convenience, and a method for the preparing the same. With a nanofibrous structure, the multi-layered anti-adhesion barrier of the present invention effectively blocks the infiltration or migration of blood and cells and promotes the healing of wounds. It is not torn or broken when folded or rolled and can be easily handled using small surgical instruments. Thus, it can minimize a foreign body reaction when used in various surgical operations.

### BACKGROUND ART

Adhesion occurs when blood flows out and is clotted during the healing of wounds caused by inflammation, gash, abrasion, surgery, etc. resulting in adhesion of neighboring organs or tissues. If cells invade the tissues, a much stronger adhesion is created.

Post-surgical adhesion is a very critical medical situation, which may result in pains, ileus, infertility, etc. Sometimes, it causes malfunction of organs or tissues, leading to another surgery or possibly loss of life. Particularly, it is reported that the rate of adhesion occurring after open surgery is as high as 60 to 95%.

As a recent method to prevent adhesion, anti-adhesion barriers are inserted during surgeries. Various types of anti-adhesion barriers in the form of a solution, gel, film, etc. are used.

The material used in the anti-adhesion barrier should be one that can function as barrier while the wound heals and is degraded thereafter. Also, the material should be free from toxicity itself and should not produce toxic substances through degradation or metabolism.

For the anti-adhesion material, bio-originated natural polymers such as polysaccharides and proteins, non-bio-originated natural polymers, water-soluble synthetic polymers, water-insoluble synthetic polymers, etc. are used. Specifically, PEG, polysaccharides [oxidized regenerated cellulose (ORC), sodium carboxymethylcellulose (CMC), dextran sulfate, sodium hyaluronate (HA), chondroitin sulfate (CS), etc.], PLA, PGA, PLGA, collagen, fibrin, etc. are used. These materials are used alone or in combination.

U.S. Pat. No. 6,599,526 discloses a pericardial anti-adhesion patch comprising a collagenous material and a non-living cellular component for preventing adhesion during surgery. U.S. Pat. No. 6,566,345 discloses anti-adhesion compositions in the form of a fluid, gel or foam made of intermacromolecular complexes of polysaccharides such as carboxyl-containing polysaccharides, polyethers, polyacids, polyalkylene oxides, etc. and synthetic polymers. Korean Patent Publication No. 2003-0055102 discloses an anti-adhesion barrier for preventing inflammation and healing wounds comprising carboxymethylcellulose (CMC) and gellan gum. But, the anti-adhesion barriers in the form of a gel, fluid, foam, etc. are not accurately fixed at the wound site; they move downward because of gravity and, thus, are less effective in healing wounds and reducing adhesion.

European Patent No. 092,733 discloses anti-adhesion barriers in the form of a membrane, gel, fiber, nonwoven, sponge, etc. prepared from crosslinking of carboxymethylcellulose (CMC) and PEO. However, carboxymethylcellulose is less biocompatible than bio-originated materials. Since polyethylene glycol or other synthetic polymers are not biodegradable, only materials having a small molecular weight and capable of being metabolized can be used. However, since materials having a small molecular weight are absorbed quickly, the role of the anti-adhesion barrier cannot be sustained sufficiently. U.S. Pat. No. 6,133,325 discloses membrane type anti-adhesion compositions made of intermacromolecular complexes of polysaccharides and polyethers.

Korean Patent Publication No. 2002-0027747 discloses that a water-soluble polymer gel prepared from alternating copolymerization of a block copolymer of p-dioxanone and L-lactide with polyethylene glycol (PEG) can be utilized as an anti-adhesion barrier, drug carrier, tissue adhesive, alveolar membrane, etc. But, this gel type anti-adhesion barrier is also problematic in accurately fixing it at such wound sites as the abdominal internal organs or tissues which are constantly moving.

U.S. Pat. No. 6,630,167 discloses an anti-adhesion barrier prepared from crosslinked hyaluronic acid. Since hyaluronic acid is a polysaccharide found in animal and human tissues, it has superior biocompatibility. However, it is degraded quickly, with a half life of only 1 to 3 days, and is problematic when used as anti-adhesion barrier. Since the crosslinked hyaluronic acid is a water-soluble polymer, its mechanical strength weakens when in contact with water because it absorbs a lot of water. There also remains the problem of removing the residuals of the crosslinking agent used to chemically crosslink hyaluronic acid in order to delay its degradation.

U.S. Pat. No. 6,693,089 discloses a method of reducing adhesion using an alginate solution and Korean Patent Publication No. 2002-0032351 discloses a semi-IPN (semi-interpenetrating network) type anti-adhesion barrier using water-soluble alginic acid and CMC, in which alginates are selectively bound to calcium ions. However, these patents are also not without the problems of quick degradation and use of non-bio-originated material.

There is a patent application about the treatment of cellulose acetate with siloxane. But, since celluloses are sensitive to pH, there is a difficulty in processing them. Also, although they are natural polymers, celluloses are not a constituent of the human body and are known to have the potential to cause a foreign body reaction. Furthermore, there remains the task of modifying their structure, e.g., through oxidation, so that they can be hydrolyzed inside the body.

Anti-adhesion barriers currently on the market are in the form of a film, sponge, fabric, gel, solution, etc. In general, the film or sponge type is easier to fix at a specific site than the solution or gel type. Interceed from Johnson & Johnson is the first commercialized anti-adhesion barrier. It is a fabric type product made of ORC and adheres tightly to highly irregular organs or tissues. But, as mentioned earlier, ORC is a non-bio-oriented material and has poor biocompatibility. Also, because of a very large pore size, cells or blood proteins may easily penetrate the barrier, and the anti-adhesion barrier is deformed by external force during handling. Seprafilm is a film type anti-adhesion barrier made of HA and CMC by Genzyme Biosurgery. However, it tends to roll when in contact with water and be brittle when it is dry. Thus, wet hands have to be avoided and moisture should be minimized at the surgical site. Especially, Seprafilm is restricted to use in laparoscopic surgery.

HYDROSORB Shield from MacroPore Biosurgery, which is used for adhesion control in certain spinal applications, or SurgiWrap from Mast Biosurgery, which is used after open surgery, are transparent film type anti-adhesion barriers made of poly(L-lactide-co-D,L-lactide) (PLA, 70:30), which is a biodegradable polymer. With a long biodegradation period of at least 4 weeks and superior mechanical strength, they are known as easy-to-handle products. Films made of PLA or poly(glycolic acid) (PGA) are easy to roll to one side, but they do not adhere well to the three-dimensionally, highly irregular surfaces of organs or tissues. Also, since these materials are hydrophobic, they do not absorb moisture well. Therefore, they do not adhere well to the wet surface of organs or tissues. Besides, when hydrolyzed in the body, they give acidic degradation products, which may cause inflammation and adhesion.

DuraGen Plus from Integra is a sponge type anti-adhesion barrier made of collagen from an animal source, which has been developed for surgery and neurosurgery. Since the collagen sponge absorbs moisture, it readily adheres to the surface of organs. However, it has relatively weak physical strength and, because of excessive moisture absorption, tends to be too heavy to handle or transport to another site. Additionally, because a material derived from an animal source is used, there is a possibility of immune rejection or exposure to animal pathogens or viruses.

Electrospinning is the technique of making nanofibers using the voltage difference between a polymer solution and a collector. This technique has the following advantages—no pollution, less waste of resources and relatively simple facilities. Electrospun nanofibers have a diameter in the range from tens to hundreds of nanometers and, thus, have a maximized surface area. The maximized surface area offers high reactivity and sensitivity.

Since nanofiber nonwovens have a random structure with numerous knots and joints, they are stronger than other materials of the same thickness. Also, with a much smaller fiber diameter, they have very superior flexibility.

There has been a lot of effort to use nanofibers in the field of medicine. For example, U.S. Pat. Nos. 6,685,956 and 6,689,374 disclose biodegradable fibrous articles for use in medical applications, in which a drug is incorporated into a composite of at least two different biodegradable polymer fibers to enable control of the drug release. However, since synthetic polymers contact tissues, a foreign body reaction or inflammation may occur. In addition, they are not effective in preventing adhesion caused by infiltration of blood or cells, because of the inability to control the pore size. U.S. Pat. No. 6,790,455 discloses a cell delivery system comprising a base layer of a fibrous matrix, a layer of cells dispersed on the base layer and a thin, porous fibrous matrix top layer for improved transportation of oxygen and nutrients. However, the intermediate cell layer may be the cause of increased adhesion because of growth and proliferation of cells in the layer.

U.S. Pat. No. 6,689,166 discloses a use of a biodegradable or non-degradable, biocompatible nonwoven nanofibril matrix as a tissue engineering device. U.S. Pat. No. 6,306,424 discloses a biodegradable composite made of a fibrous layer attached to three-dimensional porous foams for use in tissue engineering applications. However, because the tissue engineering devices have a large pore size for easier transportation of nutrients and oxygen, they may increase adhesion caused by infiltration, attachment and proliferation of cells.

U.S. Pat. No. 6,753,454 discloses a novel fiber electrospun from a substantially homogeneous mixture of a hydrophilic polymer and a weakly hydrophobic polymer for use as a dressing. But, since the hydrophilic polymer or the weakly hydrophobic polymer loses mechanical strength when swollen by water, the fiber may be deformed or torn during handling.

The foregoing techniques, in which biodegradable synthetic polymers are used, are problematic in that inflammation cannot be avoided when the polymers directly contact tissues or blood, because they are bio-originated materials. Despite the superior flexibility of nanofibers, non-hydrophilic materials do not adhere well to wet tissues, and thus are not easily fixed at a specific site.

To conclude, the conventional techniques have the problem that, since synthetic polymers are used, and although they are biodegradable, inflammation cannot be avoided when the polymers directly contact tissues or blood, because they are bio-originated materials. Also, despite the superior flexibility of nanofibers, non-hydrophilic materials do not adhere well to wet tissues, and thus are not easily fixed at a specific site. Further, the small diameter and porosity designed to improve transportation of drugs and cells or to cover the wound are not appropriate in an anti-adhesion barrier for internal organs.

In general, an anti-adhesion barrier has to satisfy the following requirements.

First, infiltration or attachment of cells or blood should be avoided through precise control of pore size or use of materials non-adherent to blood or cells. Second, the anti-adhesion barrier should be able to be attached at the desired site for a specified period of time. Third, a foreign body reaction should be minimized to reduce inflammation, which is the cause of adhesion. Fourth, the biodegradation period should be able to be controlled, so that the barrier capacity can be sustained for a requisite period of time. Fifth, the anti-adhesion barrier should be flexible and have superior mechanical properties, including tensile strength and wet strength, for ease of handling during surgery. Sixth, there should be no deformation for a necessary period of time, because the wound should be covered exactly.

Surgical operation can be divided into open surgery and laparoscopic surgery. Currently, laparoscopic surgery is on the increase because it leaves a smaller scar at the surgical site and adverse reactions to anesthesia are reduced, etc. Laparoscopic surgery is carried out by making small cuts of less than 10 mm and inserting forceps or other surgical instruments through the cuts. Since anti-adhesion barriers should be inserted in the human body through the cuts, they should not be torn or broken when folded or rolled and should be able to be moved or handled with small-sized surgical instruments.

Thus, the development of anti-adhesion barriers that can solve the problems of the conventional techniques and satisfy the afore-mentioned requirements is needed.

### DISCLOSURE

### Technical Problem

An object of the present invention is to provide a multi-layered anti-adhesion barrier having improved anti-adhesion properties by solving the problems of the conventional gel, solution, sponge, film or nonwoven type anti-adhesion systems, including adhesion to tissues or organs, flexibility, physical strength, ease of handling (ease of folding and bending), etc., offers improved user convenience, and a method for the preparing the same.

Another object of the present invention is to provide a multi-layered anti-adhesion barrier having a nanofibrous structure and, thus, being able to block the infiltration or migration of blood and cells, thereby having improved anti-adhesion properties and promoting the healing of wounds, is resistant to tearing or breaking when folded or rolled, operable or transportable with small-sized surgical instruments and, thus, applicable to various surgical operations, and a method for the preparing the same.

Still another object of the present invention is to provide a multi-layered anti-adhesion barrier that can be degraded or absorbed in the body, completely excreted out of the body after healing of the wound, handled easily and capable of minimizing a foreign body reaction in the body.

### Technical Solution

To attain the objects, the present invention provides a multi-layered anti-adhesion barrier comprising:

a) a nanofibrous structured base layer of a hydrophobic, biodegradable, biocompatible polymer; and

b) a polymer layer of a hydrophilic, bio-originated polymer.

The present invention also provides a method for preparing a multi-layered anti-adhesion barrier comprising the steps of:

a) forming a nanofibrous structured base layer by electrospinning a hydrophobic, biodegradable, biocompatible polymer; and

b) forming a polymer layer on the surface of the base layer by coating a hydrophilic, bio-originated polymer.

Hereunder is given a detailed description of the present invention.

The present inventors completed the present invention by finding out that a multi-layered anti-adhesion barrier prepared by forming a base layer with a hydrophobic, biodegradable, biocompatible polymer having superior mechanical properties and forming a polymer layer of a hydrophilic, bio-originated polymer on one or both sides of the base layer has superior flexibility and physical strength, is readily attached to complicated, wet tissues, has superior biocompatibility and, thus, is readily applicable to surgeries.

The present invention is characterized by an anti-adhesion barrier comprising a nanofibrous structured base layer of a hydrophobic, biodegradable, biocompatible polymer and a polymer layer of a hydrophilic, bio-originated polymer.

Hereunder is given a more detailed description of the anti-adhesion barrier of the present invention.

a) Base Layer

The base layer is made of a hydrophobic, biodegradable, biocompatible polymer and has a nanofibrous structure.

For the hydrophobic, biodegradable, biocompatible polymer, polypeptide, polyamino acid, polysaccharide, aliphatic polyester, poly(ester-ether), poly(ester-carbonate), polyanhydride, polyorthoester, polycarbonate, poly(amide ester), poly(α-cyanoacrylate), polyphosphazene, etc. may be used alone or in combination.

Specifically, a polypeptide such as albumin, fibrinogen, collagen, gelatin and derivatives thereof; a polyamino acid such as poly-L-glutamic acid, poly-L-leucine, poly-L-lysine and derivatives thereof; an aliphatic polyester such as poly(β-hydroxyalkanoate), polyglycolide, polylactide, polyglactin, poly(α-malic acid), poly-ε-caprolactone and derivatives thereof; a poly(ester-ether) such as poly(1,4-dioxan-2-one), poly(1,4-dioxepan-7-one) and derivatives thereof; a poly(ester-carbonate) such as poly(lactide-co-glycolide), poly(glycolide-co-13-dioxan-2-one) and derivatives thereof; a polyanhydride such as poly(sebacic anhydride)), poly[ω-(carboxyphenoxy)alkyl carboxylic anhydride] and derivatives thereof; a polycarbonate such as poly(1,3-dioxan-2-one) and derivatives thereof; a poly(amide ester) such as polydepsipeptide(poly) and derivatives thereof; a poly(α-cyanoacrylate) such as poly(ethyl α-cyanoacrylate) and derivatives thereof; a polyphosphazene and derivatives thereof, etc. can be used.

Preferably, the poly(lactide-co-glycolide) is one comprising lactide and glycolide with a proportion of 90:10 to 10:90 by molar ratio. Preferably, it has an intrinsic viscosity ranging from 0.1 to 4.0, and more preferably, from 0.2 to 2.0.

The hydrophobic, biodegradable, biocompatible polymer may be prepared into a nanofibrous structured base layer by electrospinning, where the hydrophobic, biodegradable, biocompatible polymer is used in the form of a solution or melt.

The hydrophobic, biodegradable, biocompatible polymer solution is electrospun at a concentration of 0.1 to 80 wt %, with a viscosity in the range from 50 to 1,000 cP when melted, so that the hydrophobic, biodegradable, biocompatible polymer comprises 10 to 99 wt % of the anti-adhesion barrier. More preferably, it is electrospun at a concentration of 0.5 to 50 wt %, so that the hydrophobic, biodegradable, biocompatible polymer comprises 40 to 90 wt % of the anti-adhesion barrier. If the concentration of the polymer solution is less than 0.1 wt %, fibers cannot be obtained because of insufficient viscosity. In contrast, if the concentration is more than 80 wt %, spinning does not occur or results in unstable spinning because the tension of the spinning solution overpowers the electric force due to high viscosity. In addition, if the hydrophobic, biodegradable, biocompatible polymer comprises less than 10 wt % of the anti-adhesion barrier, such physical properties as strength and elongation may be insufficient. In contrast, if it comprises more than 99 wt %, the surface coating layer for improving biocompatibility may become thin and the adherence to tissues may become weak.

The electrospinning may be carried out by the conventional electrospinning method employed to prepare nanofibers. Preferably, the electrospinning is carried out with a voltage in the range from 1 to 60 kV, a spinning distance in the range form 1 to 60 cm and a flow rate in the range from 1 to 80 μl/min, and more preferably with a voltage in the range from 5 to 40 kV, a spinning distance in the range form 5 to 45 cm and a flow rate in the range from 2 to 50 μl/min.

The resultant nanofibrous structured base layer has a nanofiber diameter preferably in the range from 10 to 5,000 nm, and more preferably in the range from 50 to 2,000 nm. The porosity is preferably in the range from 20 to 99%, and more preferably in the range from 40 to 95%. Additionally, the pore size is preferably in the range from 10 nm to 50 μm, and more preferably in the range from 50 nm to 10 μm. If the pore size is smaller than 10 nm, the adhesiveness of the base layer to the polymer layer becomes weak. In contrast if it is larger than 50 μm, cells or blood may infiltrate or migrate though the pores.

The nanofibrous structured base layer preferably has a thickness in the range from 1 to 1,000 μm, and more preferably in the range from 5 to 500 μm. If the thickness is less than 1 μm, infiltration of blood and cells cannot be blocked effectively and such physical properties as strength and elongation may be insufficient. In contrast, if is larger than 1,000 μm, the fibrous layers may be separated from one another, thereby increasing foreign body sensation and causing formation of granulation tissues.

b) Polymer Layer

The polymer layer is made of a hydrophilic, bio-originated polymer and is formed on the surface of the nano structured base layer of a hydrophobic, biodegradable, biocompatible polymer.

The bio-originated polymer may be a proteoglycan such as chondroitin sulfate, dermatan sulfate, keratan sulfate, heparan sulfate, hyaluronic acid, heparin, collagen, gelatin, elastin and fibrin; a glycoprotein such as fibronectin, laminin, vitronectin, thrombospondin and tenascin; a phospholipid such as phosphatidylcholine, phosphatidylserine, phosphatidylethanolamine, sphingomyelin and derivatives thereof; or a glycolipid such as cerebroside, ganglioside, galactocerebroside and derivatives thereof and cholesterol, etc.

The bio-originated polymer may be crosslinked to have a weight-average molecular weight in the range from thousands to millions before use, for easier handling, better control of degradation rate, etc.

The crosslinking may be carried out by the conventional crosslinking method. Specifically, an epoxide crosslinking agent, a sulfone crosslinking agent or a carbodiimide crosslinking agent may be used. In addition, such methods as radical crosslinking, anion crosslinking, cation crosslinking, plasma-induced surface activation, γ-ray irradiation, gelation using pH-dependent viscosity change, gelation by freezing/thawing, etc. may be utilized.

The epoxide crosslinking agent may be 1,4-butanediol diglycidyl ether, 1,2,7,8-diepoxyoctane, etc. The sulfone crosslinking agent may be divinyl sulfone, etc. And, the carbodiimide crosslinking agent may be 1-ethyl-3-(3-dimethylaminopropyl)carbodiimide, etc.

The crosslinked, bio-originated polymer preferably has a crosslinking density in the range from 1 to 90%, and more preferably in the range from 3 to 40%. If the crosslinking density is less than 1% or more than 90%, the desired convenience in handling, control of degradation rate, etc. cannot be fully attained.

The bio-originated polymer or the crosslinked bio-originated polymer preferably comprises 1 to 80% of the anti-adhesion barrier, and more preferably 3 to 60 wt %. If the content of the bio-originated polymer or the crosslinked bio-originated polymer is less than 1 wt %, it is not uniformly coated on the surface of the hydrophobic nanofiber and the adhesivity to tissues is reduced. In contrast, if the content is more than 60 wt %, the final product has poor flexibility and physical strength.

The bio-originated polymer or the crosslinked bio-originated polymer is coated on the surface of the base layer to form a polymer layer. Of course, the coating of the bio-originated polymer may be carried out by the common methods such as electrospinning, casting, dip coating, spray coating, etc.

The bio-originated polymer or the crosslinked bio-originated polymer may be coated on top of the base layer to prepare a double-layered anti-adhesion barrier or may be coated on top and bottom of the base layer to prepare a triple-layered anti-adhesion barrier (see FIG. 1). If required, the anti-adhesion barrier may be prepared into more than three layers.

The polymer layer preferably has a thickness in the range from 0.1 to 500 μm, and more preferably in the range from 1 to 200 μm. If the thickness is less than 0.1 μm, the anti-adhesion barrier has poor adhesivity and biocompatibility. In contrast, if it is more than 500 μm, the anti-adhesion barrier cannot be folded or rolled well and, thus, is less applicable in laparoscopic surgery.

The anti-adhesion barrier of the present invention, which comprises a nano structured base layer of a hydrophobic, biodegradable, biocompatible polymer and a polymer layer of a hydrophilic, bio-originated polymer formed on the base layer, has a tensile strength of at least 2.0 N/mm² and superior flexibility and physical strength. When applied to the tissues of a wound site, it readily adheres to the tissues as the bio-originated polymer layer absorbs moisture and swells. And, with superior biocompatibility, the anti-adhesion barrier can reduce inflammation and offers improved anti-adhesion effects by blocking migration of blood and cells through the pores.

The source materials of the anti-adhesion barrier are free from toxicity and are not harmful to the human body. While the wound healing, they function as a physical barrier to prevent adhesion of the tissues or organs and, when the healing is completed, they are degraded in the body and absorbed, metabolized or excreted out of the body. The degradation period may be changed by controlling the surface area/volume ratio of the base layer, the composition of the polymers, the presence or absence of a crystal structure, the thickness of the polymer layer, and the crosslinking density. However, it is preferable that the degradation period is within 28 days.

The anti-adhesion barrier may further comprise a drug commonly used in the preparation of a conventional anti-adhesion barrier. The drug may be added during the preparation of the anti-adhesion barrier or just before the application to a wound site. The drug may be thrombin, aprotinin, etc. for promoting early hemostasis; a steroidal or non-steroidal anti-inflammatory agent; heparin for preventing thrombosis; tissue plasminogen activator, etc.

Besides the use as an anti-adhesion barrier during and after surgery, the multi-layered anti-adhesion barrier of the present invention may also be used as a wound dressing, tissue engineering scaffold, cell carrier, etc.

The present invention also provides a method for preparing a multi-layered anti-adhesion barrier comprising the steps of forming a nanofibrous structured base layer by electrospinning a hydrophobic, biodegradable, biocompatible polymer and forming a polymer layer on the base layer by coating a hydrophilic, bio-originated polymer.

The base layer is formed by the electrospinning method commonly employed in the preparation of conventional nanofibers. The electrospinning is preferably carried out with a voltage in the range from 1 to 60 kV, a spinning distance in the range from 1 to 60 cm and a flow rate in the range from 1 to 80 μl/min, and more preferably with a voltage in the range from 5 to 40 kV, a spinning distance in the range from 5 to 45 cm and a flow rate in the range from 2 to 50 μl/min.

Preferably, the resultant base layer has a pore size in the range from 10 nm to 50 μm, and more preferably in the range from 50 nm to 10 μm. In addition, the base layer preferably has a thickness in the range from 1 to 1,000 μm, and more preferably in the range from 5 to 500 μm. If the thickness is smaller than 1 μm, infiltration of blood and cells cannot be blocked effectively and the anti-adhesion barrier will not have superior physical properties. In contrast, if it is larger than 1,000 μm, the fibrous layers may be separated from one another, thereby increasing foreign body sensation and causing formation of granulation tissues.

The polymer layer may be coated on the base layer by such conventional coating methods as electrospinning, casting, dip coating, spray coating, etc. The polymer layer may be coated on top of the base layer to prepare a double-layered anti-adhesion barrier or may be coated on top and bottom of the base layer to prepare a triple-layered anti-adhesion barrier. If required, the anti-adhesion barrier may be prepared into more than three layers.

The polymer layer preferably has a thickness in the range from 0.1 to 500 μm, and more preferably in the range from 1 to 200 μm. If the thickness is less than 0.1 μm, the anti-adhesion barrier may have poor adhesiveness and biocompatibility. In contrast, if is more than 500 μm, the anti-adhesion barrier becomes hard and brittle, making it resistant to modification and less applicable to laparoscopic surgery.

### ADVANTAGEOUS EFFECTS

The multi-layered anti-adhesion barrier of the present invention can solve the problems of conventional gel, solution, sponge, film or nonwoven type anti-adhesion systems, including adhesion to tissues or organs, flexibility, physical strength, ease of handling (ease of folding and bending), etc., offers improved user convenience and a method for the preparing the same. With a nanofibrous structure, the multi-layered anti-adhesion barrier of the present invention effectively blocks the infiltration or migration of blood and cells and promotes the healing of wound. It is not torn or broken when folded or rolled and can be easily handled using small surgical instruments. Thus, it can minimize foreign body reaction when used in various surgical operations.

### DESCRIPTION OF DRAWINGS

FIG. 1 schematically illustrates the multi-layered anti-adhesion barrier of the present invention.

FIG. 2 schematically illustrates the electrospinning apparatus used in the present invention.

FIG. 3 is an SEM micrograph of the polylactide electrospun in accordance with the present invention.

FIG. 4 is a micrograph of the polylactide electrospun in accordance with the present invention.

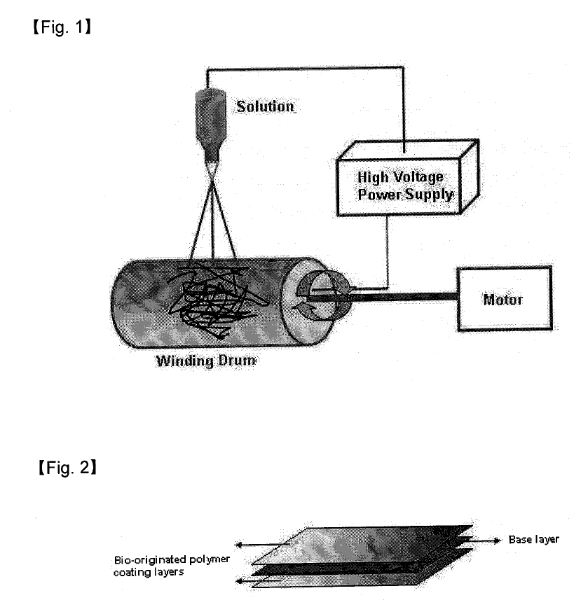

### BEST MODE

Practical and preferred embodiments of the present invention are illustrated as shown in the following examples. However, it will be appreciated that those skilled in the art may, in consideration of this disclosure, make modifications and improvements within the spirit and scope of the present invention.

### Examples 1 to 9

### Formation of Nanofibrous Structured Base Layers

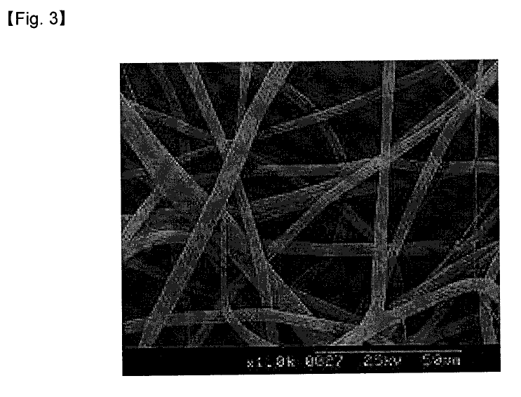

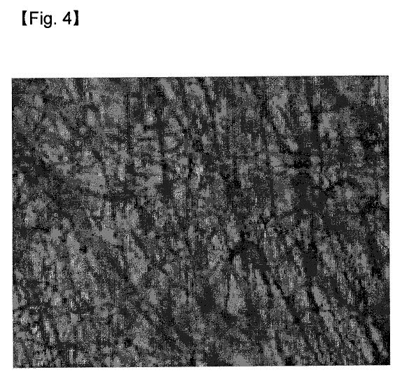

Nanofibrous structured base layers were formed with different hydrophobic, biodegradable, biocompatible polymers, concentrations, electrospinning voltages, electrospinning distances and flow rates, as shown in Table 1 below. The electrospinning apparatus illustrated in FIG. 2 was used. The SEM micrograph and micrograph of the polylactide electrospun in Example 5 are shown in FIG. 3 and FIG. 4, respectively.

TABLE 1

| | | | Electrospinning condition | | |
|---|---|---|---|---|---|
| | | Concentration | | Distance | Flow rate |
| Example # | Polymers | (wt %) | Voltage (kV) | (cm) | (ml/hour) |
| 1 | Poly-L-glutamic acid | 6 | 10 | 5 | 1 |
| | | 8 | 15 | 10 | |
| | | 10 | 18 | 15 | |
| 2 | Poly(1,3-dioxan-2-one) | 6 | 10 | 5 | 1 |
| | | 8 | 15 | 10 | |
| | | 10 | 20 | 15 | |
| 3 | Polydepsipeptide | 6 | 10 | 10 | 1 |
| | | 8 | 15 | 15 | |
| | | 10 | 20 | 20 | |
| 4 | Poly(sebacic anhydride) | 5 | 20 | 10 | 1 |
| | | 10 | 30 | 15 | |
| 5 | Polylactide | 2 | 15 | 10 | 1 |
| | | 5 | 20 | 15 | |
| | | 10 | 25 | 20 | |
| 6 | Polyglycolide | 6 | 10 | 5 | 1 |
| | | 8 | 15 | 10 | |
| | | 10 | 18 | 15 | |
| 7 | Polylactide-co-glycolide | 6 | 10 | 5 | 1 |
| | | 8 | 15 | 10 | |
| | | 10 | 18 | 15 | |
| 8 | Poly(1,4-dioxan-2-one) | 2 | 10 | 5 | 1 |
| | | 3 | 15 | 10 | |
| | | 5 | 20 | 15 | |
| | | 8 | 25 | 20 | |
| 9 | Polyphosphazene | 2 | 10 | 5 | 1 |
| | | 5 | 15 | 10 | |
| | | 10 | 20 | 15 | |

In general, fiber diameter and physical properties of nanofibers are determined by the polymer concentration, spinning voltage, spinning distance and flow rate. The nanofiber diameter becomes smaller when the polymer concentration is smaller, the spinning voltage is higher and the spinning distance is larger.

As seen in Table 1, when poly(1,3-dioxan-2-one) was used (Example 2), a fiber structure was attained at the concentration of 8 to 10 wt % because of superior fiber-forming ability. Spinning was possible even at the low voltage of 10 to 20 kV. When polydepsipeptide was used (Example 3), a continuous fiber structure without beads was attained at the voltage of 15 to 20 kV, when the spinning distance was adjusted to 15 cm. When polylactide and polyglycolide were used (Examples 5 and 6), a fiber structure was attained at the concentration of 5 wt % or higher. The best result was obtained at the concentration of 8 wt %, at the voltage of 25 kV and 20 kV and at the spinning distance of 15 cm. The nanofiber had a diameter in the range from hundreds to thousands of nanometers. And, when polylactide-co-glycolide was used (Example 7), different fiber-forming ability was displayed at different molecular weight. The best mechanical properties were attained at the concentration of 8 wt %.

### Examples 10 to 18

### Preparation of Multi-Layered Anti-Adhesion Barriers

Multi-layered anti-adhesion barriers were prepared by coating a bio-originated polymer selected from polylactide-co-glycolide, poly ε-caprolactone, polylactide and hyaluronic acid on the nanofibrous structured base layers prepared in Examples 1 to 9 with different coating methods (see Table 2 below). Electrospinning was carried out using the electrospinning apparatus illustrated in FIG. 2 and a spinning solution in which the bio-originated polymer was dissolved at a voltage of 10 to 40 kV. Dip coating was carried out by dip coating the bio-originated polymer solution and drying the anti-adhesion barrier in an oven of 70° C. Casting was carried out by coating the bio-originated polymer solution on the base layer, casting it into a film and drying the anti-adhesion barrier. Spray coating was carried out by spraying the bio-originated polymer solution on the base layer and drying the anti-adhesion barrier in an oven of 70° C. for 24 hours.

TABLE 2

| | | | Bio-originated polymers | | | |
|---|---|---|---|---|---|---|
| | Nanofiber | Thickness | | | Thickness | |
| Example # | base layer | (μm) | Polymer | Content (%) | (μm) | Coating method |
| 10 | Example 1 | 60 | Hyaluronic | 40-50 | 50 | Electrospinning |
| | | | acid | 59-60 | | Dip coating |
| | | | | 50-60 | | Casting |
| | | | | 50 | 40 | Spray coating |
| 11 | Example 2 | 60 | Heparin | 40-50 | 50 | Electrospinning |
| | | | | 59-60 | | Dip coating |
| | | | | 59-60 | | Casting |
| | | | | 50 | 40 | Spray coating |
| 12 | Example 3 | 60 | Keratan | 40-50 | 50 | Electrospinning |
| | | | sulfate | 59-60 | | Dip coating |
| | | | | 59-60 | | Casting |
| | | | | 50 | 40 | Spray coating |
| 13 | Example 4 | 60 | Hyaluronic | 40-50 | 50 | Electrospinning |
| | | | acid | 59-60 | | Dip coating |
| | | | | 59-60 | | Casting |
| | | | | 50 | 40 | Spray coating |
| 14 | Example 5 | 60 | Collagen | 40-50 | 50 | Electrospinning |
| | | | | 59-60 | | Dip coating |
| | | | | 59-60 | | Casting |
| | | | | 50 | 40 | Spray coating |
| 15 | Example 6 | 60 | Heparin | 40-50 | 50 | Electrospinning |
| | | | | 59-60 | | Dip Coating |
| | | | | 59-60 | | Casting |
| | | | | 50 | 40 | Spray coating |
| 16 | Example 7 | 60 | Gelatin | 40-50 | 50 | Electrospinning |
| | | | | 59-60 | | Dip coating |
| | | | | 59-60 | | Casting |
| | | | | 50 | 40 | Spray coating |
| 17 | Example 8 | 60 | Collagen | 40-50 | 50 | Electrospinning |
| | | | | 59-60 | | Dip coating |
| | | | | 59-60 | | Casting |
| | | | | 50 | 40 | Spray coating |
| 18 | Example 9 | 60 | Hyaluronic | 40-50 | 50 | Electrospinning |
| | | | acid | 59-60 | | Dip coating |
| | | | | 59-60 | | Casting |
| | | | | 50 | 40 | Spray coating |

As seen in Table 2, dip coating and casting offered improved mechanical strength compared to when nanofiber was used alone. Spray coating enabled coating with a coating solution having a smaller viscosity. And, electrospinning enabled a thinner coating.

### Example 19

Poly(lactide-co-glycolide) (PLGA) having a lactide/glycolide ratio of 70:30 was dissolved in chloroform to 2 wt % and electrospun to form a nano structured base layer having a thickness of 60 μm. Subsequently, hyaluronic acid (HA) was dissolved in distilled water to 1 wt %, adjusted to pH 1.5 with 1 N HCl, uniformly coated on the nano structured base layer by casting to form a polymer layer having a thickness of 50 μm thickness. The procedure of freezing at −20° C. for 22 hours and thawing at 25° C. for 2 hours repeated twice. A multi-layered anti-adhesion barrier was obtained following neutralization with PBS, washing and freeze drying.

### Example 20

Dissolved HA was coated on the nano structured base layer of PLGA prepared in Example 19 and dried to prepare a PLGA/HA film. Subsequently, 1-ethyl-3-(3-dimethylaminopropyl)carbodiimide (EDAC), which is the crosslinking agent for HA, was added to a 90:10 (w/w) mixture of ethanol and water. The PLGA/HA film was immersed in the resultant solution and dried to obtain a multi-layered anti-adhesion barrier.

### Example 21

To HA dissolved in 0.5% NaOH was added 1,4-butanediol diglycidyl ether (BDDE) as a crosslinking agent. The solution was coated on the nano structured base layer of PLGA prepared in Example 19. After reaction at 5° C. for 16 hours, unreacted BDDE was removed. A multi-layered anti-adhesion barrier was obtained following dialysis, filtration and freeze drying.

A tensile strength test was performed for the multi-layered anti-adhesion barriers prepared in Examples 19 and 20 using a 25 kgf load cell. Crosshead speed was adjusted to 6 mm/min and grip distance was fixed at 20 mm. The results are given in Table 3.

TABLE 3

Example #

| | 19 | 20 |
|---|---|---|
| Width (mm) | 10 | 10 |
| Thickness (mm) | 0.111 | 0.146 |
| Maximum tensile strength (gf/mm²) | 111.803 | 550.610 |
| Maximum elongation (%) | 38.398 | 5.939 |

As seen in Table 3, when HA was crosslinked with 1-ethyl-3-(3-dimethylaminopropyl)carbodiimide (Example 20), tensile strength was improved by about 5 times than when it was not crosslinked (Example 19).

An animal test was performed using the multi-layered anti-adhesion barriers prepared in Examples 10 to 21. All of them showed superior adhesiveness to wound tissues and organs during surgery and consistent adhesiveness even at irregular sites. They contributed to quick healing of wounds and were excreted completely out of the body after the healing.

### INDUSTRIAL APPLICABILITY

The multi-layered anti-adhesion barrier of the present invention can solve the problems of the conventional gel, solution, sponge, film or nonwoven type anti-adhesion systems, including adhesion to tissues or organs, flexibility, physical strength, ease of handling (ease of folding and bending), etc., offers improved user convenience. With a nanofibrous structure, the multi-layered anti-adhesion barrier of the present invention effectively blocks the infiltration or migration of blood and cells and promotes the healing of wounds. It is not torn or broken when folded or rolled and can be easily handled using small surgical instruments. Thus, it can minimize a foreign body reaction when used in various surgical operations.

Those skilled in the art will appreciate that the concepts and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the present invention as set forth in the appended claims.

1. A multi-layered anti-adhesion barrier comprising:
a) a nanofibrous structured base layer of a hydrophobic, biodegradable, biocompatible polymer; and
b) a polymer layer of a hydrophilic, bio-originated polymer.

2. The multi-layered anti-adhesion barrier as set forth in claim 1, wherein a) the hydrophobic, biodegradable, biocompatible polymer is at least one selected from the group consisting of polypeptide, polyamino acid, polysaccharide, aliphatic polyester, poly(ester-ether), poly(ester-carbonate), polyanhydride, polyorthoester, polycarbonate, poly(amide ester), poly(α-cyanoacrylate) and polyphosphazene.

3. The multi-layered anti-adhesion barrier as set forth in claim 1, wherein a) the hydrophobic, biodegradable, biocompatible polymer is a nanofibrous structured base layer prepared by electrospinning.

4. The multi-layered anti-adhesion barrier as set forth in claim 1, wherein a) the hydrophobic, biodegradable, biocompatible polymer comprises 10 to 99 wt % of the anti-adhesion barrier.

5. The multi-layered anti-adhesion barrier as set forth in claim 1, wherein a) the base layer has a nanofiber diameter in the range from 10 to 5,000 nm, a porosity in the range from 20 to 99% and a pore size in the range from 10 nm to 50 μm.

6. The multi-layered anti-adhesion barrier as set forth in claim 1, wherein a) the base layer has a thickness in the range from 1 to 1,000 μm.

7. The multi-layered anti-adhesion barrier as set forth in claim 1, wherein b) the bio-originated polymer is at least one selected from the group consisting of chondroitin sulfate, dermatan sulfate, keratan sulfate, heparan sulfate, hyaluronic acid, heparin, collagen, gelatin, elastin, fibrin, fibronectin, laminin, vitronectin, thrombospondin, tenascin, phosphatidylcholine, phosphatidylserine, phosphatidylethanolamine, sphingomyelin and derivatives thereof, cerebroside, ganglioside, galactocerebroside and derivatives thereof, and cholesterol.

8. The multi-layered anti-adhesion barrier as set forth in claim 1, wherein b) the bio-originated polymer is crosslinked using an epoxide crosslinking agent, a sulfone crosslinking agent or a carbodiimide crosslinking agent or by radical crosslinking, anion crosslinking, cation crosslinking, plasma-induced surface activation, γ-ray irradiation, gelation using pH-dependent viscosity change or gelation by freezing/thawing.

9. The multi-layered anti-adhesion barrier as set forth in claim 8, wherein the crosslinking is carried out using at least one crosslinking agent selected from the group consisting of an epoxide crosslinking agent, a sulfone crosslinking agent and a carbodiimide crosslinking agent.

10. The multi-layered anti-adhesion barrier as set forth in claim 8, wherein the crosslinked bio-originated polymer has a crosslinking density in the range from 1 to 90%.

11. The multi-layered anti-adhesion barrier as set forth in claim 1, wherein b) the bio-originated polymer comprises 1 to 80 wt % of the anti-adhesion barrier.

12. The multi-layered anti-adhesion barrier as set forth in claim 1, wherein b) the bio-originated polymer is coated on the base layer by electrospinning, casting, dip coating or spray coating.

13. The multi-layered anti-adhesion barrier as set forth in claim 1, wherein b) the polymer layer is formed on top of the base layer, or on top and bottom of the base layer.

14. The multi-layered anti-adhesion barrier as set forth in claim 1, wherein b) the polymer layer has a thickness in the range from 0.1 to 500 μm.

15. The multi-layered anti-adhesion barrier as set forth in claim 1, which has a tensile strength of at least 2.0 N/mm².

16. The multi-layered anti-adhesion barrier as set forth in claim 1, which further comprises at least one drug selected from the group consisting of thrombin, aprotinin, steroidal, non-steroidal anti-inflammatory agent, heparin and tissue plasminogen activator.

17. A method for preparing the multi-layered anti-adhesion barrier as set forth in claim 1, which comprises the steps of:
a) forming a nanofibrous structured base layer by electrospinning a hydrophobic, biodegradable, biocompatible polymer; and
b) forming a polymer layer by coating a hydrophilic, bio-originated polymer on the base layer.

18. The method for preparing a multi-layered anti-adhesion barrier as set forth in claim 17, wherein the electrospinning in the step a) is carried out with a voltage in the range from 1 to 60 kV, a spinning distance in the range from 1 to 60 cm and a flow rate in the range from 2 to 80 μl/min.

19. The method for preparing a multi-layered anti-adhesion barrier as set forth in claim 17, wherein the coating in step b) is carried out by electrospinning, casting, dip coating or spray coating.
